(19) United States
(12) Patent Application Publication
(10) Pub. No.: US 2007/0185521 A1
(43) Pub. Date: Aug. 9, 2007

(54) Rapid exchange assembly

(75) Inventors: Bao Bui, Sherbrooke (CA); Arman H. Valaie, Bloomington, IN (US); John A. Brumleve, Bloomington, IN (US); Daniel J. Sirota, Bloomington, IN (US); Robert M. Eells, Bloomington, IN (US); Sarah E. Waite, Cory, IN (US)

Correspondence Address: BRINKS HOFER GILSON & LIONE/INDY/COOK, ONE INDIANA SQUARE, SUITE 1600, INDIANAPOLIS, IN, 46204-2033, US

(73) Assignee: Cook Incorporated, Bloomington, IN (US)

(21) Appl. No.: 11/633,246

(22) Filed: Dec. 4, 2006

Related U.S. Application Data
(60) Provisional application No. 60/742,285, filed on Dec. 5, 2005.

(51) Int. Cl. A61M 29/00

(52) U.S. Cl. 606/191

(57) ABSTRACT

A rapid exchange assembly comprises an introducer sheath, and a dilator receivable within a lumen of the introducer sheath. The dilator has an open distal end and a lumen extending therein. A distal portion of the dilator extends distally beyond the distal end of the introducer sheath when the dilator is received in the lumen of the introducer sheath. The dilator has an opening through a wall in the distal portion which communicates with the open distal end of the dilator. A wire guide is receivable through the open distal end of the dilator and extends through the opening in the distal portion of the dilator outwardly of the assembly along the length of the introducer sheath.

### RELATED APPLICATION

The present patent document claims the benefit of the filing date under 35 U.S.C. §119(e) of Provisional U.S. Patent Application Ser. No. 60/742,285, filed Dec. 5, 2005, which is hereby incorporated by reference.

### BACKGROUND

1. Technical Field

The present invention relates to medical devices, and more particularly, to a medical device rapid exchange assembly.

2. Background Information

In recent years, rapid exchange devices have gained increased acceptance in the medical arts. Such devices are now routinely used in common medical procedures, such as, for example, percutaneous transluminal coronary angioplasty (PTCA). When a rapid exchange device is used in a PTCA procedure, a guide catheter is normally initially introduced into the patient's arterial system, e.g., at the groin area. The guide catheter is advanced through the arteries to a location near the patient's heart. A wire guide is inserted into the guide catheter and advanced to the distal end of the guide catheter, at which point it is steered to extend beyond the guide catheter and through the stenosis. The proximal end of the wire guide, which extends externally of the patient, is then inserted (i.e., “back loaded”) through the wire guide lumen of an angioplasty balloon catheter at the distal tip of the catheter. The balloon catheter is advanced until the wire guide exits the balloon catheter through an opening in the body of the angioplasty balloon catheter in the distal tip region. A working fluid is then pumped through the balloon catheter, thereby inflating the balloon and dilating the passage through the stenosis.

Since the wire guide of a rapid exchange device need not extend through the entire lumen of the balloon catheter, it is not necessary to utilize an extremely long wire guide when the angioplasty catheter is to be exchanged with another catheter. Exchange wires that are required for such operations in conventional over-the-wire systems must normally be about twice the length of the catheter. This length not only requires additional manpower, but also introduces sanitation concerns, since the proximal end of the wire extends an excessive distance beyond the proximal end of the catheter. On the other hand, with a rapid exchange catheter, a single operator may normally perform the exchange procedure, since the proximal end of the wire guide extends only a short distance, if at all, beyond the proximal end of the catheter. Rapid exchange catheters are further described in many prior art documents, such as U.S. Pat. Nos. 5,451,233 and 6,371,961, incorporated herein by reference.

Although rapid exchange technology has been successfully used with angioplasty and like procedures, the use of this technology has not been applied in many other areas of medical practice in which benefits can be obtained. For example, it would be desirable to utilize this technology in procedures in which it is desired to introduce, or remove, a solid or fluid through a catheter. Examples of such procedures include an embolectomy procedure and a thrombectomy procedure. During such procedures, a catheter is introduced into the vasculature to enable removal of one or more emboli or thrombi through the catheter. Catheters of this type are normally threaded into the vasculature over a wire guide. However, since the wire guide occupies a portion of the lumen of the removal device, this portion of the lumen is not available for passage therethrough of the solid or fluid matter. This is disadvantageous in situations when it is desired to provide as large a passage area as possible. When large emboli are present, for example, some of the emboli may not be removable through the available area of the lumen. Similarly, when liquid medicaments are to be introduced, it is also desired to provide as large a cross sectional area as possible in the introducing sheath or device, so that the greatest possible volume of the medicament can be introduced in a single application. When the wire guide occupies a portion of the cross section of the lumen, the area available for further medical use is thus compromised.

It would be desirable to provide an assembly for introducing and/or removing a substance from a body vessel wherein the available space of the lumen of the introducing or removal device is not reduced due to the presence of a wire guide or other structure that occupies a portion of the lumen. Similarly, it would be desirable to expand the use of rapid exchange technology by providing a dilator assembly that is amenable to a rapid exchange operation.

### BRIEF SUMMARY

The present application addresses the limitations of the prior art. In one form, the invention comprises a rapid exchange assembly. The rapid exchange assembly comprises an introducer device, and a dilator receivable within a lumen of the introducer device. The dilator has a proximal end, an open distal end and a lumen extending therein. A distal portion of the dilator extends distally beyond the distal end of the introducer device when the dilator is received in the lumen of the introducer device. The dilator has an opening through a wall in the distal portion, which opening communicates with the open distal end of the dilator. A guiding device, such as a ramp, may be provided at the proximal end of the dilator opening for guiding a wire guide that has been inserted through the distal end of the dilator outwardly of the assembly through the dilator opening.

In another form thereof, the invention comprises a method for removing a thrombus from a body vessel. A rapid exchange assembly is provided. The rapid exchange assembly includes an introducer sheath having a proximal end, a distal end and a lumen extending therein. A dilator is receivable within the lumen of the introducer sheath. The dilator has a proximal end, an open distal end and a lumen extending therein, wherein a distal portion of the dilator extends distally beyond the distal end of the introducer sheath when the dilator is received in the lumen of the sheath. The dilator has an opening through a wall in the dilator distal portion. A wire guide has a length at least as long as the length of the introducer sheath, and has a diameter such that the wire guide is receivable through the open distal end of the dilator. An opening is formed in the body vessel, and the distal end of the wire guide is inserted into the body vessel opening. The wire guide is advanced therealong in the vessel until it substantially reaches said thrombus. The proximal end of the wire guide is inserted through the open distal end of the dilator, and passed through a portion of the dilator lumen such that it exits the dilator through the dilator opening. The rapid exchange assembly is advanced over the wire guide substantially to an area of the thrombus. The wire guide and the dilator may be disengaged by effecting relative movement therebetween until the distal end of said wire guide exits the dilator opening. The dilator is then withdrawn in the proximal direction from the lumen of the introducer sheath; and the thrombus is withdrawn through the introducer sheath lumen, e.g., by aspiration. If desired, a new sheath and dilator assembly can then be reinserted over the wire guide, or alternatively, the original sheath and/or the original dilator can be re-used and re-advanced over the wire to perform another aspiration of thrombus.

### BRIEF DESCRIPTION OF THE DRAWINGS

FIG. 1 illustrates one embodiment of a rapid exchange apparatus of the present invention, shown positioned in a body vessel;

FIG. 2 illustrates a top view of the rapid exchange dilator assembly of FIG. 1;

FIG. 3 illustrates a side view of the assembly of FIG. 1;

FIG. 4 illustrates a longitudinal cross-sectional view of the dilator assembly of FIGS. 1-3;

FIG. 5 is a top view of the distal portion of the dilator, partially in section;

FIG. 6 illustrates an alternative embodiment of a sheath having an angled distal portion; and

FIGS. 7 and 8 illustrate the use of the inventive rapid exchange assembly in removing a thrombus from a body vessel.

### DETAILED DESCRIPTION OF THE DRAWINGS AND THE PRESENTLY PREFERRED EMBODIMENTS

For the purposes of promoting an understanding of the principles of the invention, reference will now be made to the embodiments illustrated in the drawings, and specific language will be used to describe the same. It should nevertheless be understood that no limitation of the scope of the invention is thereby intended, such alterations and further modifications in the illustrated device, and such further applications of the principles of the invention as illustrated therein being contemplated as would normally occur to one skilled in the art to which the invention relates.

In the following discussion, the terms “proximal” and “distal” will be used to describe the opposing axial ends of the assembly, as well as the axial ends of various component features. The term “proximal” is used in its conventional sense to refer to the end of the assembly (or component thereof) that is closest to the operator during use of the assembly. The term “distal” is used in its conventional sense to refer to the end of the assembly (or component thereof) that is initially inserted into the patient, or that is closest to the patient.

The inventive rapid exchange assembly may be used, among other possible uses, as a rapid exchange dilator. In such use, the assembly comprises an introducer device in combination with a dilation device. Following use of the introducer/dilator combination to dilate a vessel opening, the dilator may be removed from the introducer and the remaining portions of the assembly may be used in the desired medical procedure. Non-limiting examples of such procedures include removal of emboli or thrombi from the vasculature, and the introduction or removal of solids and/or fluids from a body vessel.

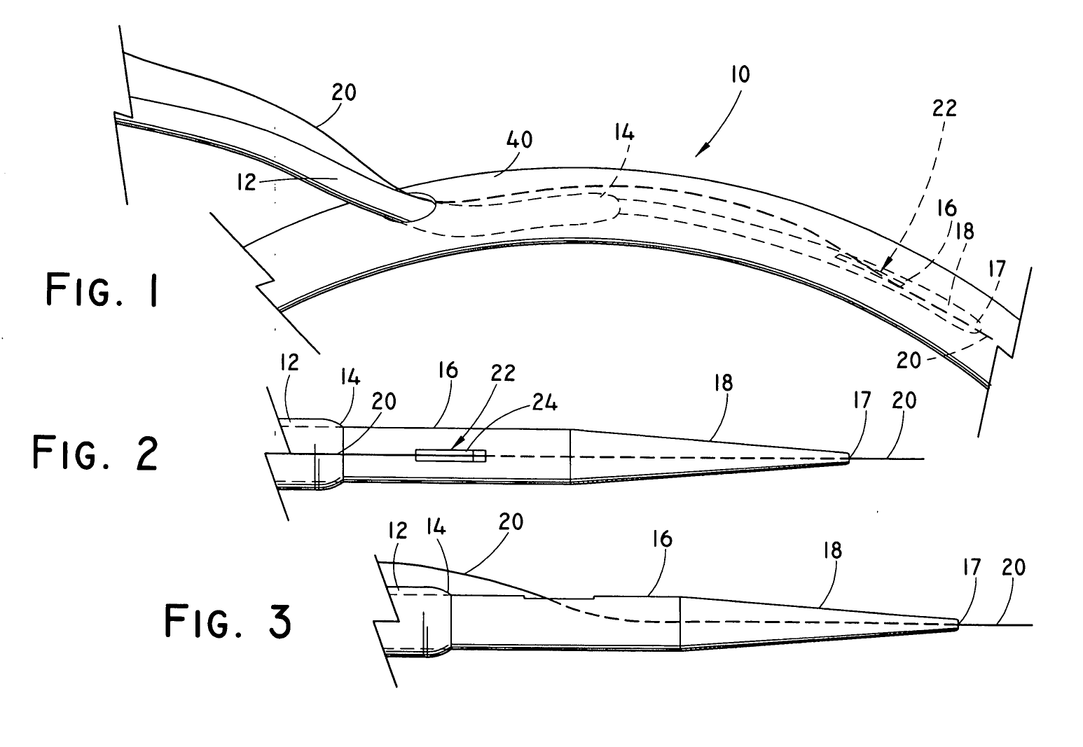

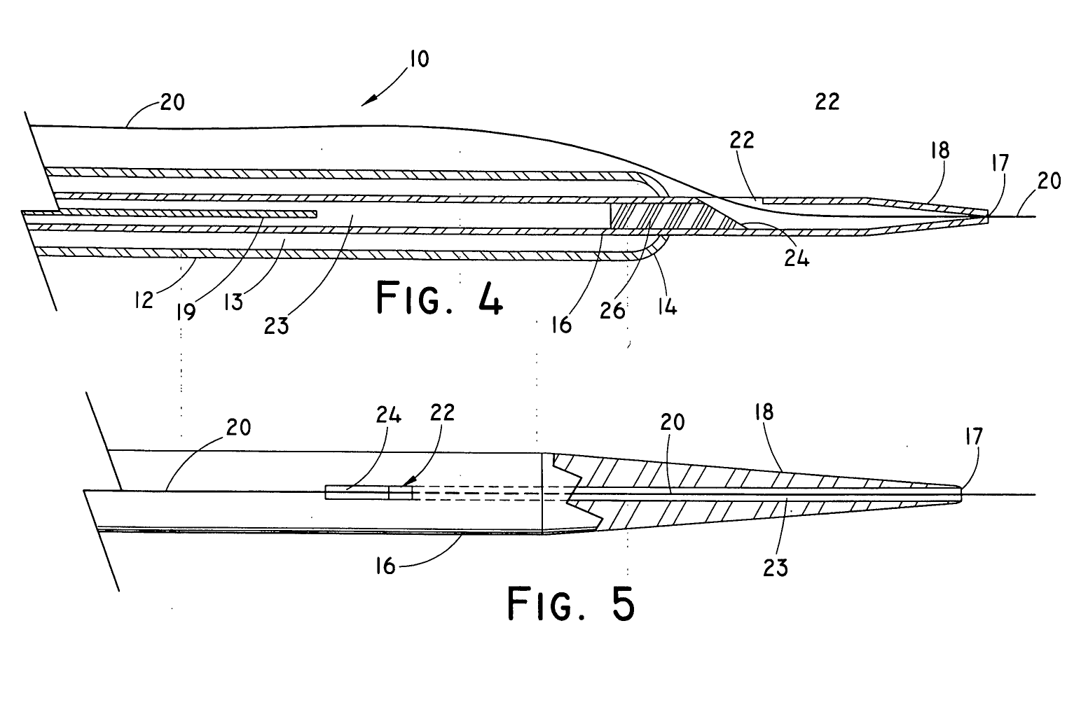

FIG. 1 illustrates the distal portion of a rapid exchange assembly 10 in accordance with one embodiment of the present invention. In this figure, the distal portion of assembly 10 is shown positioned in a body vessel 40, such as a blood vessel. FIGS. 2-5 illustrate the distal portion of the rapid exchange assembly 10 removed from the body vessel. FIG. 2 illustrates a top view of the distal portion of the rapid exchange assembly 10, and FIG. 3 illustrates a side view of the assembly. FIG. 4 illustrates a longitudinal cross-sectional view of the distal portion of the assembly. FIG. 5 is a top view of the distal portion of dilator 16, partially in section. The proximal portion of rapid exchange assembly 10 is conventional for rapid exchange catheters and devices, and need not be shown in the figures to provide an understanding of the inventive features of the assembly.

In the embodiment shown, rapid exchange assembly 10 comprises a conventional introducer device, such as introducer sheath 12, having a central lumen 13 extending therethrough. A dilator 16 extends longitudinally through central lumen 13. Dilator 16 is sized such that a distal portion 18 of dilator 16 extends beyond the distal end 14 of introducer sheath 12. A wire guide 20 runs generally parallel to introducer sheath 12 along the greater part of the exterior surface of sheath 12, in a manner generally similar to that of known rapid exchange assemblies. Wire guide 20 runs interiorly of rapid exchange assembly 10 through a portion of a lumen 23 that extends through dilator 16. In particular, wire guide 20 extends in lumen 23 through an opening 22 that extends through the wall of dilator distal portion 18. (FIG. 4).

If desired, a stiffening member 19, such as a stiffening wire or rod, may extend through at least a portion of central lumen 13 of sheath 12. When present, stiffening member 19 will generally terminate at a desired location within sheath central lumen 13. The stiffening member is preferably made from stainless steel or any other material having the desired stiffness. Stiffening members are conventional, and those skilled in the art may readily select a stiffening member of a desired length and composition for a particular application. When a stiffening member is utilized to assist in initial insertion of the introducer sheath, the stiffening member need not be retained in the sheath following insertion. Removal of the stiffening member may be desirable to enable use of the cross sectional area of the introducer lumen otherwise occupied by the stiffening member during an introduction or removal operation.

As stated, dilator 16 is provided with an opening 22 through the dilator wall. Opening 22 communicates with inner lumen 23 of the dilator. In a preferred embodiment, dilator 16 also includes an element for guiding the proximal end of wire guide 20 through dilator lumen 23 and opening 22 to the exterior of the assembly. Preferably, the guiding element comprises a plug-like structure, such as ramp 24. Those skilled in the art will appreciate that alternate structures may be readily substituted for ramp 24. When present, ramp 24 facilitates passage of the wire guide from dilator lumen 23 to the exterior of the apparatus, in a manner to be described. Among other advantages, the presence of ramp 24 reduces the bend in wire often associated with rapid exchange devices. As a result, less force is required to slide the apparatus along the wire guide.

Those skilled in the art will appreciate that there are numerous ways of cutting or otherwise forming ramp 24 in dilator 16. For example, the portion of the dilator at which the ramp is to be cut may be plugged and sealed. For convenience, the material used to form the plug 26 may be the same material used to form the dilator. In this case, an angled sideport may be initially formed through a wall in the dilator, e.g., utilizing a conventional hole punch. A piece of beading (solid tubing) is preferably cut at an angle as shown to form ramp 24. The beading is inserted into the lumen of the dilator, and directed to an appropriate position, as shown in FIG. 4. The beading may then be mechanically fixed to the dilator by conventional affixation means; for example by heating the assembly to form a heat bond between the beading and the inner diameter of the dilator, or by adhesion. In the preferred embodiment shown, the ramp portion 24 of plug 26 has a length at least about one-half the length of opening 22.

The particular location of the ramp on a particular dilator may be varied based on the intended use of the dilator, as well as any wire guide size requirements. Preferably, the distance between the distal end of opening 22 and the distal tip 17, referred to as the “tracking portion” of the dilator, is at least about twice the length of the ramp. Generally, the tracking portion is at least about 1.5 inches (3.8 cm) long. Preferably, the distance between the distal end of the introducer sheath and the proximal end of the opening, referred to as the “clearing portion” of the dilator, is at least about one-half as long as the ramp. Generally, the clearing portion is at least about one-half inch (1.3 cm) in length.

Those skilled in the art will appreciate that these lengths are only examples of possible lengths, and may be varied in a particular case. Dilators of any French size may be utilized, as long as the dilator is compatible with the particular introducer sheath and wire guide. Those skilled in the art are readily able to match a dilator with an introducer sheath and a wire guide for a particular rapid exchange application. A preferred range of dilator size for many applications is 3 to 7 French, with 5 French being a particularly preferred size. However, larger dilator sizes, such as 12 French and larger, may also be utilized for a particular application.

Introducer devices are well known in the art, and the inventive rapid exchange assembly may utilize any conventional introducer device. Introducer devices, such as introducer sheaths, are provided in a wide variety of lengths, compositions, and diameters. In addition, introducer sheaths may have a single durometer along the entire length of the sheath, or may comprise two or more segments of different durometer. Furthermore, the sheaths may optionally be provided with one or more reinforcing members, such as a coil or a braid, that extend at least partially along the length of the sheath, and may additionally comprise one or more coaxial layers. Those skilled in the art can readily select a suitable sheath for a particular application without undue experimentation.

One example of a suitable introducer sheath is the FLEXOR® introducer, available from Cook Incorporated, of Bloomington, Ind. The FLEXOR® introducer comprises an inner layer of a fluorocarbon, such as PTFE, a coil reinforcement, and an outer layer formed of a polymer, such as nylon. The outer layer is bonded to the inner layer through the turns of the coil. The FLEXOR® sheath comprises a plurality of segments of different durometer, ranging from a higher durometer proximal segment to a lower durometer distal segment. The FLEXOR® sheath is commonly provided in a set that also includes a dilator and a wire guide.

As an alternative embodiment, the sheath can be provided with a second lumen, or with an external coupling region, for the wire guide to track through so that the dilator can be removed without the wire being uncoupled from the sheath. As still another embodiment, the distal portion of the sheath can be oriented at an angle between about 10° and 20°, and more preferably, an angle of about 15° with respect to the remainder of the sheath. This arrangement may facilitate aspiration of a thrombus against the sidewalls of the vessel. One non-limiting example of a sheath 60 having an angled distal portion 62 is shown in FIG. 6.

The dilator of the present invention may be of any conventional composition. One particularly preferred composition is a lubricous fluoropolymer composition, such as PTFE. However, a significant difference between a conventional dilator, such as that commonly used in combination with the FLEXOR® sheath, and the dilator of the inventive rapid exchange assembly is that the dilator of the inventive assembly is provided with the rapid-exchange structure discussed previously and as shown in the figures.

The sheath and dilator assembly may have any length appropriate for the intended use. In most cases, the entire sheath and dilator combination will be between about 65 and 90 cm in length, although those skilled in the art will appreciate that longer, and shorter, lengths may be appropriate for a particular application. A conventional radiopaque marker band may be placed at the distal end portion of the sheath, and/or at a location on the surface of the dilator in the vicinity of the opening (such as proximal to the opening).

Wire guides are very well known in the art, and those skilled in the art can readily select an appropriate wire guide for a particular use. Non-limiting examples of suitable wire guides include elongated wire and a coil type wire guide. One particularly preferred type of wire guide is a floppy tip wire guide, such as the ROADRUNNER® wire guide, available from Cook Incorporated, of Bloomington, Ind. The wire guide may also be provided with a radiopaque marker, such as at its distal tip. In this way, the clinician is readily able to confirm under fluoroscopy that uncoupling of the wire guide from the dilator had occurred, so that the dilator may be removed from the sheath.

When a conventional introducer apparatus, such as the FLEXOR® sheath, is introduced into a body passageway, a dilator is typically positioned in the lumen of the introducer sheath. Generally, both the proximal and the distal ends of the dilator extend outwardly beyond the corresponding proximal and distal ends of the sheath. Unlike the present rapid exchange arrangement, in the conventional design the wire guide extends fully through the lumen of the dilator.

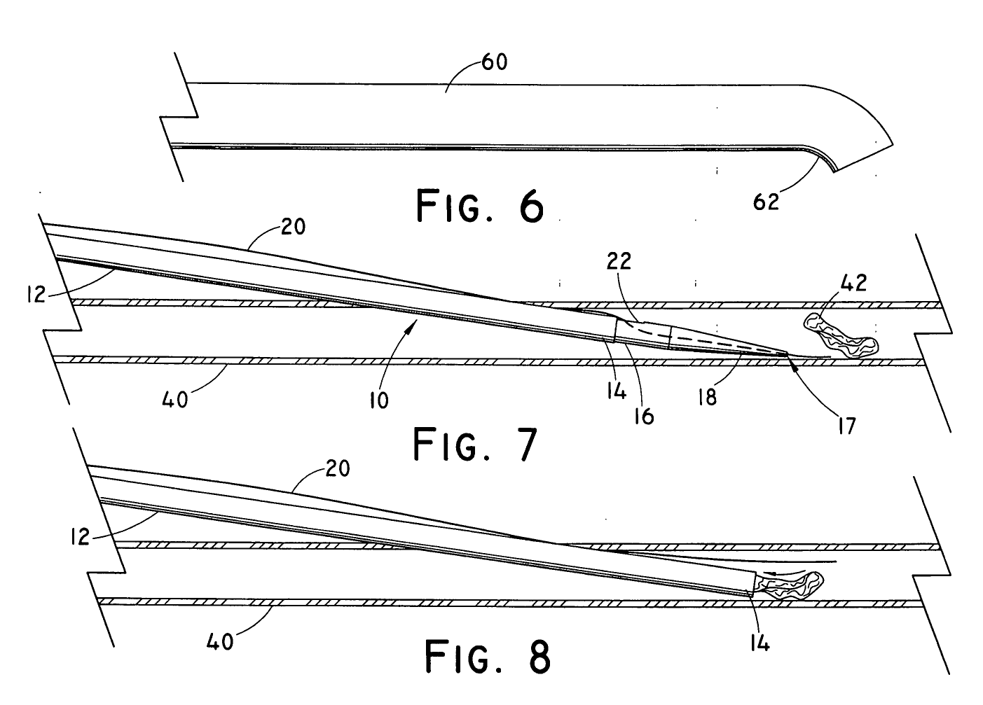

Use of a preferred embodiment of the inventive rapid exchange assembly will now be described in one of its intended uses, namely for performing a thrombectomy in a thrombosed artery or vein of a patient. This is best illustrated in FIGS. 7 and 8.

In this embodiment, the invention rapid exchange assembly comprises introducer sheath 12 and dilator 16. The dilator is disposed within the central lumen of the introducer sheath in convention fashion, such that dilator distal portion 18 extends, e.g., about four to six inches (10.2 to 15.2 cm) beyond the distal tip 14 of the introducer. Dilator 16 includes opening 22 through a side wall distal to introducer distal tip 14, as previously described. Wire guide 20 may be provided as part of the rapid exchange assembly, or separately.

Initially, the wire guide is positioned at the target site in the vessel 40 by conventional means, such as the well-known Seldinger percutaneous insertion technique. The proximal end of the wire guide, which extends externally of the patient, is then inserted through the distal tip 17 of the dilator, and advanced until it exits through the opening 22 in the side wall of the dilator. The sheath and dilator are advanced over the wire guide until they reach the proposed treatment site. This is shown in FIG. 7. The dilator is then disengaged from the wire guide, and removed from the sheath. In one possible manner of disengagement, the indwelling portion of the wire guide may be partially withdrawn until remote uncoupling of the wire guide and the dilator occurs at the treatment site. Stated another way, the wire guide is withdrawn until the distal tip of the wire guide exits through the opening of the dilator. In another possible manner of disengagement, the dilator can simply be advanced until it disengages the wire guide. In either event, following disengagement of the dilator and the wire guide, the dilator may be withdrawn through the lumen of the sheath. The wire guide can then be re-advanced in the vessel.

Removal of the dilator enables the entire lumen of introducer sheath 12 to be available for the aspiration of a thrombus 42 from the site and along the path of withdrawal from the body. This arrangement is shown in FIG. 8. The thrombus may then be aspirated through the sheath by conventional means. After aspiration from the patient, the collected thrombus can be flushed from the sheath. A new sheath and dilator assembly can then be reinserted over the wire guide, or alternatively, the original sheath and/or the original dilator can be re-used and re-advanced over the wire to perform another aspiration of thrombus. This allows for multiple passes over the same, short wire guide to remove thrombi without necessitating that the sheath track over the wire as it is being withdrawn from the patient. Without remote uncoupling as described, the dilator cannot be removed from the sheath to allow aspiration without a long wire exchange being used.

It is therefore intended that the foregoing detailed description be regarded as illustrative rather than limiting, and that it be understood that it is the following claims, including all equivalents, that are intended to define the spirit and scope of this invention.

1. A rapid exchange assembly, comprising:
an introducer device, said introducer device having a proximal end, a distal end and a lumen extending therein; and
a dilator receivable within the lumen of the introducer device, said dilator having a proximal end, an open distal end and a lumen extending therein, a distal portion of said dilator extending distally beyond the distal end of said introducer device when said dilator is received in said introducer device lumen, said dilator having an opening through a wall in said distal portion wherein said opening communicates with said open distal end of said dilator.

2. The rapid exchange assembly of claim 1, wherein said dilator opening has a proximal end and a distal end, said dilator further comprising a plug disposed at said proximal end of said opening.

3. The rapid exchange assembly of claim 2, wherein said plug has a proximal end and a distal end, said distal end comprising a ramp.

4. The rapid exchange assembly of claim 3, wherein said ramp has a length in the axial direction at least about one-half a length of said opening.

5. The rapid exchange assembly of claim 4, wherein the distal end of the opening is spaced at least about 1.5 inches (3.8 cm) from the distal end of the dilator.

6. The rapid exchange assembly of claim 5, wherein said dilator has an outer diameter of between about 3 and 12 French.

7. The rapid exchange assembly of claim 2, wherein said plug has a ramp-like leading end, and said plug is affixed to an inside diameter of said dilator.

8. The rapid exchange assembly of claim 7, wherein said plug is affixed to said inside diameter by at least one of heat bonding and adhesion.

9. The rapid exchange assembly of claim 1, wherein at least a portion of said introducer distal end is oriented at an angle of about 10 to 20° with respect to said introducer proximal end.

10. The rapid exchange assembly of claim 1, further comprising a wire guide, said wire guide having a proximal end and a distal end, said wire guide having a diameter such that the wire guide is receivable through said open distal end of said dilator and extendable through said opening in said distal portion of said dilator.

11. The rapid exchange assembly of claim 10, wherein said wire guide has a length at least as long as a length of said introducer device.

12. The rapid exchange assembly of claim 11, wherein said dilator opening has a proximal end and a distal end, said dilator further comprising a plug disposed at said proximal end of said opening, said plug having a leading end defining a ramp, said ramp being structured and arranged for facilitating passage of said wire guide through said opening from said dilator open distal end.

13. The rapid exchange assembly of claim 12, wherein said plug and said dilator are formed of a common composition.

14. The rapid exchange assembly of claim 1, further comprising a stiffening member disposed within at least a portion of said lumen of said introducer device.

15. A method for removing a thrombus from a body vessel, comprising:
providing a rapid exchange assembly, said rapid exchange assembly, comprising: an introducer sheath having a proximal end, a distal end and a lumen extending therein; a dilator receivable within the lumen of the introducer sheath, the dilator having a proximal end, an open distal end and a lumen extending therein, wherein a distal portion of the dilator extends distally beyond the distal end of said introducer sheath when the dilator is received in the lumen of the introducer sheath, the dilator having an opening through a wall in the dilator distal portion; and a wire guide having a proximal end and a distal end, said wire guide having a length at least as long as a length of said introducer sheath, and having a diameter such that the wire guide is receivable through said open distal end of said dilator;
forming an opening in said body vessel;
inserting the distal end of said wire guide into said body vessel opening, and advancing said wire guide distal end in said vessel until it substantially reaches said thrombus;
inserting the proximal end of the wire guide through the open distal end of the dilator, and passing the proximal end of said wire guide through a portion of said dilator lumen such that said proximal end of said wire guide exits said dilator through said opening;
advancing said rapid exchange assembly over the wire guide substantially to an area of said thrombus;
disengaging the wire guide and the dilator by effecting relative movement therebetween until the distal end of said wire guide exits said dilator opening;
withdrawing said dilator in the proximal direction from the lumen of said introducer sheath; and
withdrawing said thrombus through said introducer sheath lumen.

16. The method of claim 15, wherein said relative movement comprises partially withdrawing said wire guide until the distal end of said wire guide exits said dilator opening, and wherein said thrombus is withdrawn through said introducer sheath lumen by aspiration.

17. The method of claim 15, wherein said dilator opening has a proximal end and a distal end, said dilator further comprising a ramp disposed at said proximal end of said dilator opening for facilitating passage of said wire guide proximal end through said opening.

18. The method of claim 15, further comprising the step of re-advancing the wire guide in the vessel following withdrawal of the dilator.

19. The method of claim 18, further comprising the steps of reforming said rapid exchange assembly by re-inserting said dilator in said introducer sheath lumen; advancing said re-formed rapid exchange assembly over the wire guide substantially to an area of another thrombus; disengaging the wire guide and the dilator by effecting relative movement therebetween until the distal end of said wire guide exits said dilator opening; withdrawing said dilator of said re-formed rapid exchange assembly in the proximal direction from the lumen of said introducer sheath; and withdrawing said other thrombus through said introducer sheath lumen.

20. The method of claim 18, further comprising the step of providing a replacement rapid exchange assembly wherein at least one of said introducer sheath and dilator is replaced with a new introducer sheath or dilator following withdrawal of the thrombus, advancing said replacement rapid exchange assembly over the wire guide substantially to an area of another thrombus; disengaging the wire guide and the dilator by effecting relative movement therebetween until the distal end of said wire guide exits said dilator opening; withdrawing said dilator of said replacement rapid exchange assembly in the proximal direction from the lumen of said introducer sheath; and withdrawing said other thrombus through said introducer sheath lumen.
